(19) United States
(12) Patent Application Publication
(10) Pub. No.: US 2021/0169526 A1
(43) Pub. Date: Jun. 10, 2021

(54) EXTERNAL FIXATION DEVICE

(71) Applicant: Flower Orthopedics Corporation, Horsham, PA (US)

(72) Inventors: Oliver BURCKHARDT, Philadelphia, PA (US); Kevin RZASA, Wilmington, DE (US)

(21) Appl. No.: 17/269,206

(22) PCT Filed: Aug. 16, 2020
(86) PCT No.: PCT/US2019/046745

§ 371 Date: Feb. 17, 2021

Related U.S. Application Data
(60) Provisional application No. 62/719,184, filed on Aug. 17, 2018.

(51) Int. Cl. A61B 17/62

(52) U.S. Cl. CPC A61B 17/62

(57) ABSTRACT

The present application discloses embodiments related to an external bone fixation device configured to correct bone deformities or repair bone injuries. The device comprises of at least two expandable fixator rings which are arranged at a distance to each other and plurality of adjustable length struts connecting the two expandable fixator rings configured to be adjustable to change the position and orientation of the expandable fixator rings.

### CROSS-REFERENCE TO RELATED APPLICATIONS

This application claims priority to U.S. provisional application No. 62/719,184, filed Aug. 17, 2018, the contents of which are incorporated herein in their entirety.

### BACKGROUND OF THE INVENTION

Orthopedic external fixation devices are used in many orthopedic treatments to fixate, distract, compress, or reduce bone segments and to correct deformities. Generally, fixation devices or fixators are used to stabilize fractured bone pieces and to facilitate the healing of bones at a bone repair site. Such fixators can be used by orthopedic surgeons to restore the patient's anatomy at a fracture following an injury or distract an osteotomy site in bone lengthening or shortening procedures. One type of external fixation devices comprises two base members connected by one or more adjustable struts that can be adjusted to set the two base members into a desired relative configuration that is appropriate for treating a particular orthopedic fracture, nonunion, malunion, or deformities of skeletal bone fragments. The base members usually have ring-like structures and can be full rings or partial rings.

In one conventional example disclosed in U.S. Pat. No. 6,030,386 to Taylor et al., the adjustable telescoping struts connect two ring-like structures via conventional universal-joints that provide a variety of attachment angles. However, the conventional ring like structures do not provide the ability to expand while in use.

There is a need in the art for improved size-adjustable orthopedic external fixation devices. The present invention solves this problem.

### SUMMARY OF THE INVENTION

In one aspect, the present invention relates to an expandable external fixator device comprising: a rigid frame having a ring-like shape and a plurality of pins positioned on the frame; and a plurality of rigid segments, each segment having at least two slots connected to at least two pins of the frame and slidable between an inner position and an outer position; wherein the device has a variable size defined by the position of the plurality of segments.

In one embodiment, each rigid segment comprises a plurality of through holes. In one embodiment, the rigid frame has a ring-like shape that is substantially circular, ovoid, horseshoe, square, and rectangular. In one embodiment, the position of each rigid segment is lockable by a mechanism selected from the group consisting of: a screw, a clamp, an adhesive, a weld, a ratchet, a knob, a latch, and a pin.

In another aspect, the present invention relates to an expandable external fixator device comprising: a plurality of interlocking rigid sections, each section having a slot positioned at a first end and a tab positioned at a second end; wherein the tab of each section is sized to fit within the slot of each section, and wherein the device has a variable size defined by the position of each tab within each slot.

In one embodiment, each section comprises a plurality of through holes. In one embodiment, the plurality of sections are configured to interlock to form a shape that is substantially circular, ovoid, horseshoe, square, and rectangular. In one embodiment, the position of each rigid segment is lockable by a screw or clamp. In one embodiment, the clamp comprises snap-in barbs configured to engage an interconnected tab and slot. In one embodiment, the clamp comprises hinged pins configured to engage an interconnected tab and slot. In one embodiment, the clamp comprises lockable pins configured to engage an interconnected tab and slot.

In another aspect, the present invention relates to an external fixation system comprising: one or more expandable fixator devices of the present invention; and one or more length-adjustable struts; wherein each expandable fixator device is securable to each other by the one or more length-adjustable struts.

In one embodiment, the system further comprises one or more treads or rollers.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1B, depicts a top view of exemplary expandable rings. FIG. 1A depicts an exemplary expandable ring in a contracted form with a diameter 15. FIG. 1B depicts an exemplary expandable ring with an expanded diameter 17.

FIG. 2, comprising FIG. 2A through FIG. 2C, depicts a top view of exemplary expandable rings. FIG. 2A depicts an exemplary expandable ring in a contracted form with a width 19. FIG. 2B depicts an exemplary expandable ring with an expanded width 21. FIG. 2C depicts an exemplary ring segment, having a male and female end to interconnect

FIG. 3, comprising FIG. 3A through FIG. 3B, depicts an exemplary fixator ring with attachment points. FIG. 3A depicts a top view of an exemplary ring with four attachment points. FIG. 3B depicts a side view of the exemplary ring with the attached ring section to the attachment point.

FIG. 4 depicts an exemplary expandable ring with connector devices used to interconnect the ring segments together.

FIG. 5, comprising FIG. 5A through FIG. 5B, depicts an exemplary snap in connector. FIG. 5A depicts a side view of the snap-in connector. FIG. 5B, depicts a bottom view of the snap in connector.

FIG. 6, comprising FIG. 6A through FIG. 6C, depicts an exemplary hinged connector. FIG. 6A, depicts a side view of the exemplary hinged connector. FIG. 6B depicts a top view of the exemplary hinged connector after deployment. FIG. 6C depicts a side view of the exemplary hinged connector before deployment.

FIG. 7, comprising FIG. 7A through FIG. 7B, depicts an exemplary pin connector. FIG. 7A depicts a side view of the exemplary pin connector before deployment. FIG. 7B depicts a side view of the exemplary pin connector after deployment.

FIG. 8 depicts exemplary ring segments assembled with connector devices to fasten additional instruments to the ring segments.

FIG. 9 depicts an exemplary pre-built configuration of an external fixator system.

### DETAILED DESCRIPTION

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

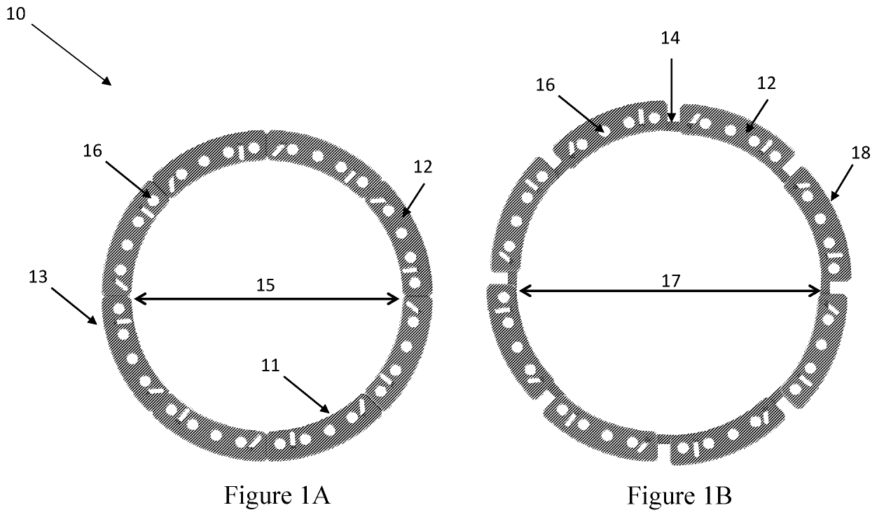

FIG. 1 is a top view of an expandable fixator ring 10 of the present invention. The expandable fixator ring 10 includes a top surface 12, a bottom surface (not shown), an inner side surface 11, and an outer side surface 13. The top surface 12 includes plurality of segments movable on a circular frame 14, each segment having slots that are slidable along pins on frame 14 to increase or decrease inner diameter 15. Each segment can be lockable using any suitable mechanism commonly used in the art. The locking mechanism can include, but is not limited to: a screw, a clamp, an adhesive, a weld, a ratchet, a knob, a latch, a pin, and the like. In one exemplary embodiment, the diameter of the expandable fixator ring before expansion is between 100-150 mm. In one exemplary embodiment, the diameter of the expandable fixator ring after full expansion is between 200-250 mm. The top surface 12 includes plurality of holes 16 that extend through the top surface 12 to the bottom surface (not shown). The holes 16 may be used for attachment of wire and half pin fixation elements (bolts), threaded or telescopic connection rods, plates, posts, or other device connection elements a surgeon may deem necessary for connecting to the expandable fixator ring 10.

Such devices, for example, include bone-pin retaining devices and bone-wire retaining devices. Due to the substantially flat surface of the expandable fixator ring 10 and the plurality of holes 16, a user is provided significant flexibility in appropriately placing the bone-pin retaining devices, bone-wire retaining devices or any other device recommended by the surgeon at desired locations. Thus, a user can couple any of these devices at numerous locations around the circumference of each of the expandable fixator rings 10 as well as coupling the devices at the top surface 12 or lower surface of the expandable fixator ring 10. Devices that can be used to facilitate interaction between the fixator device and portions of a bone include, for example, a series of bone-wires and bone-pins.

Expandable fixator ring 10 may be made of metal (e.g., stainless steel, titanium, etc.), composites (e.g., Carbon PEEK, etc.), or any other material deemed suitable. Although described as a ring, the expandable fixator ring 10 can include any shape that allows at least one adjustable device to be releasably connected to it. For example. The expandable fixator ring 10 can be a circle shape, oval shape, horseshoe shape, square shape, rectangle shape, or any other shape deemed suitable. In one exemplary embodiment, the expandable fixator ring 10 is planar creating a relatively flat surface.

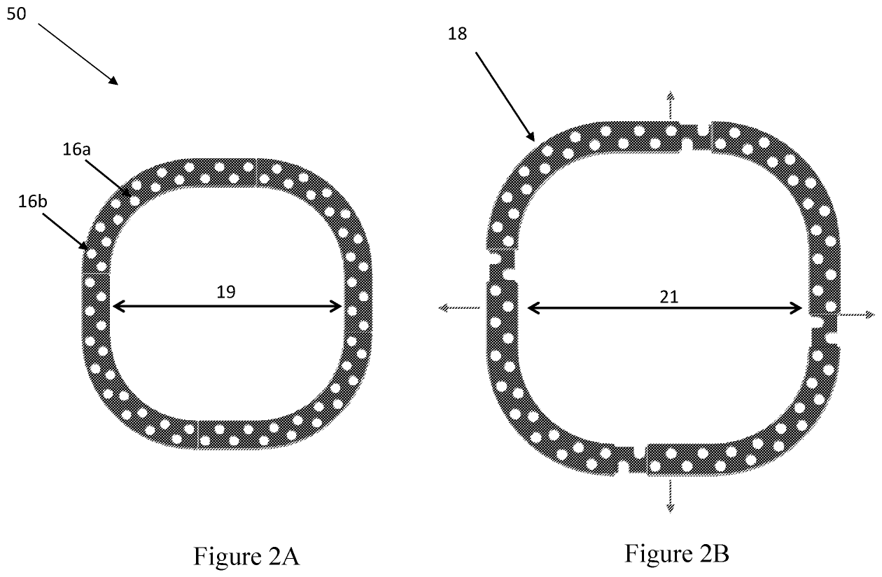

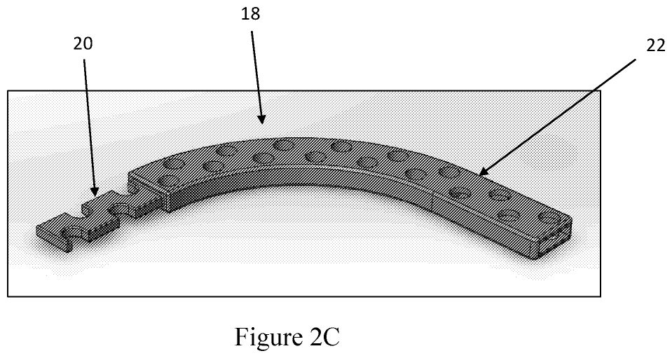

FIG. 2 depicts another exemplary fixator ring 50, comprising a plurality of ring interlocking sections 18.

In one exemplary embodiment, the expandable fixator ring 50 is composed of several ring interlocking sections 18. FIG. 2C, depicts one exemplary embodiment of the ring interlocking section 18. In one exemplary embodiment, the ring interlocking section 18, each have a male 20 and female 22 ends to interconnect. Each interlocking section 18 can be lockable using any suitable mechanism commonly used in the art. The locking mechanism can include, but is not limited to: a screw, a clamp, an adhesive, a weld, a ratchet, a knob, a latch, a pin, and the like. The ring interlocking section 18 can be adjusted to expand the inner diameter of the ring. In one exemplary embodiment, the diameter of the expandable fixator ring before expansion is between 100-150 mm. In one exemplary embodiment, the diameter of the expandable fixator ring after full expansion is between 200-250 mm.

In one embodiment, the plurality of holes 16 can be threaded, unthreaded, or a combination of threaded and unthreaded such that the holes 16 are configured to receive both locking and non-locking fasteners. In one exemplary embodiment, as depicted in FIG. 2, holes 16 are arranged in two rows 16a and 16b. This spacing allows for utilization of consecutive holes and adds versatility to the design. The number and placement of the holes 16 is largely a matter of design choice.

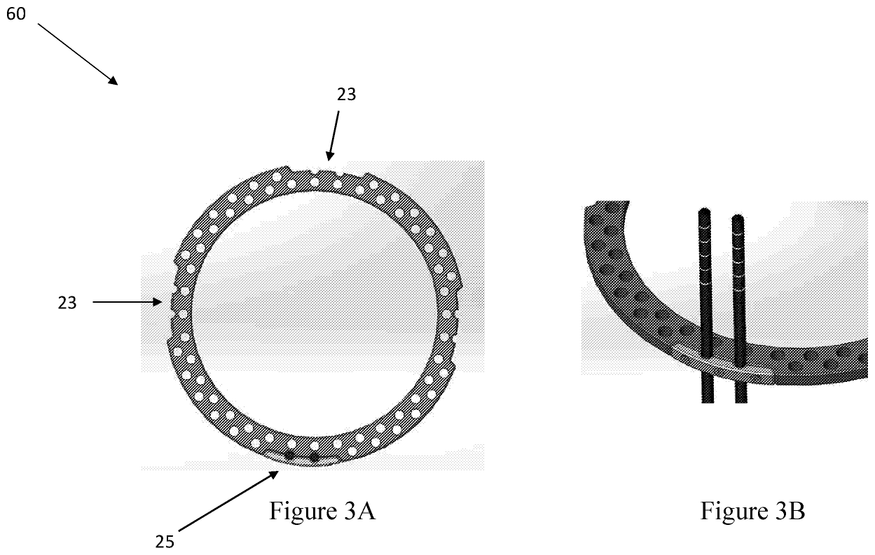

FIG. 3 depicts another exemplary configuration of the expandable fixator ring 60. As shown in FIG. 3A, in one exemplary embodiment, the fixator ring 60 can further include four attachments points 23. As shown in FIG. 3B, in one exemplary embodiment, a ring attachment section 25 can be attached to any of the attachment points 23 to secure a wire and half pin fixation elements (bolts), threaded or telescopic connection rods, plates, posts, or other device connection elements a surgeon may deem necessary for connecting to the expandable fixator ring 60.

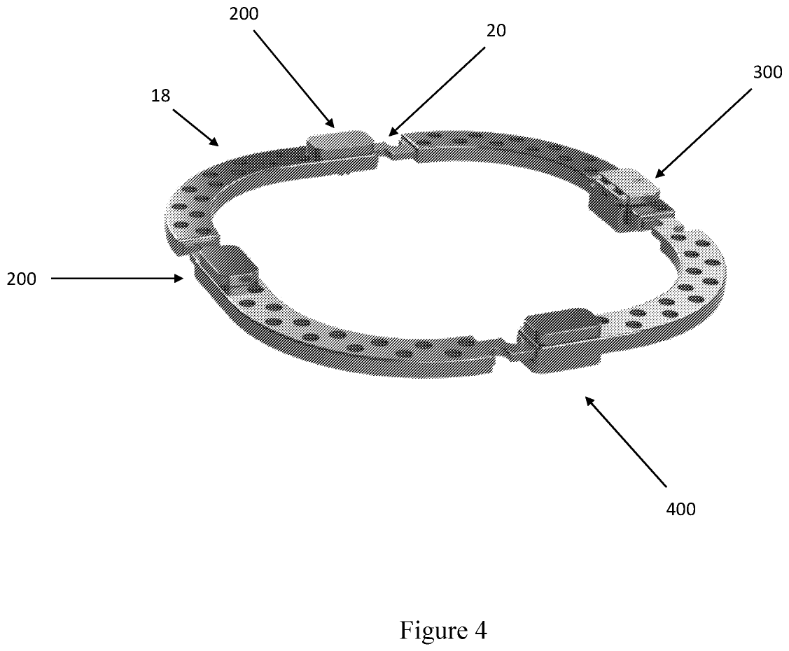

FIG. 4 depicts a side view of the expandable fixator ring 10 wherein the ring is composed of four ring interlocking section 18, connected through a connector in an expanded configuration.

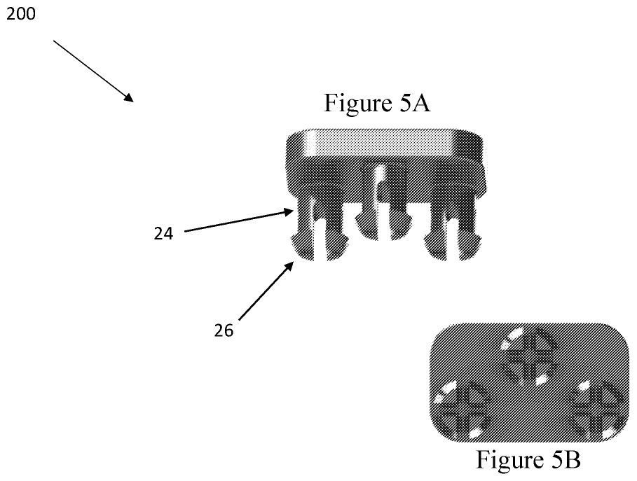

In one exemplary embodiment, the connector may be a snap-in connector 200. As depicted in FIG. 5, snap in connector comprises an arm 24 with an orthogonally extending barbed shaft 26 located at the end of the extending arms 24. Barbs 26 allow for easy insertion of the shaft into the channel but, like fishhooks, resist removal therefrom.

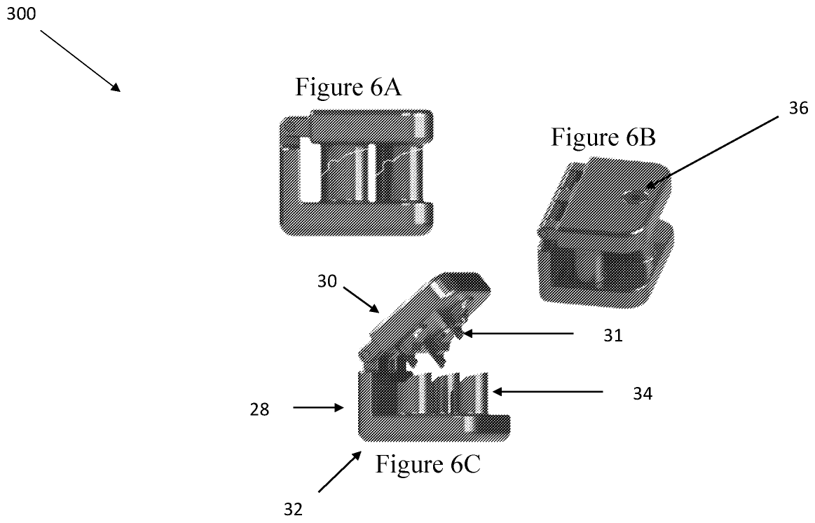

In another exemplary embodiment, the connector may be a hinged connector 300. As depicted in FIG. 6, the hinge mechanism comprises a fixed hinge component 28 and a moveable hinge component 30. The fixed hinge component comprises a base portion 32 with at least one longitudinally extending pin portion 34. The extending pin or pins 34 are sized to fit through holes 16 and attaches to one side of an expandable fixator ring 10. The moveable hinge component 30 comprises of a structure 31, attached to the underside of the moveable hinge 30 that mates with the pin/pins 34 and locks/secures the connector with a set screw 36.

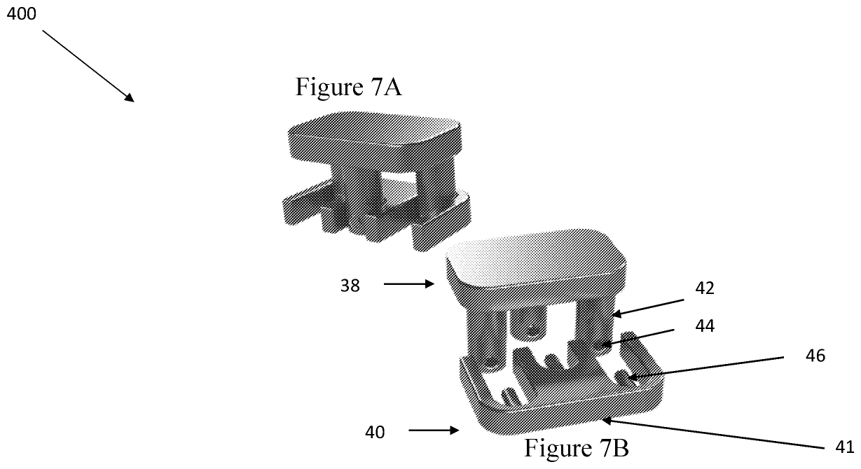

In another exemplary embodiment, the connector may be a pin connector 400. As depicted in FIG. 7, the pin connector comprises a top 38 and bottom portion 40. The top portion 38 comprises at least one extending arm 42 with a hole at the end 44. The bottom portion 40 comprises a base 41 with extending pins 46. The extending arms 42 are sized to fit through holes 16 of the expandable fixator ring 10 and can be secured by mating with the extending pins 46 of the bottom portion 40.

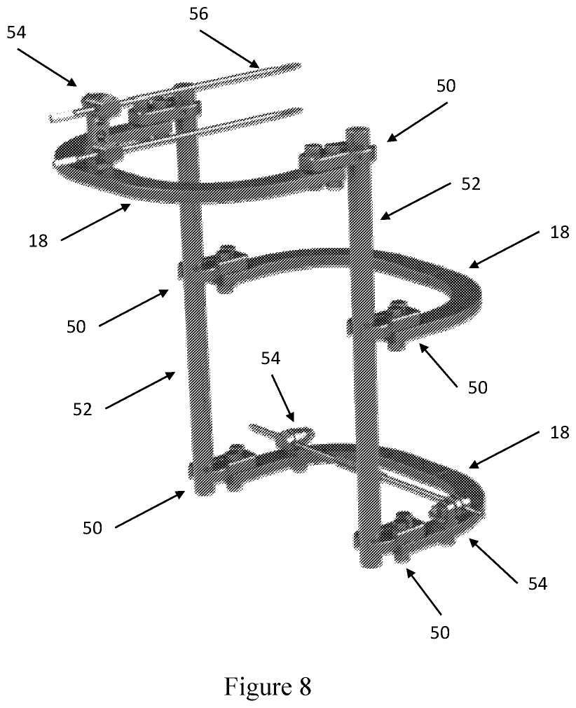

FIG. 8 shows a plurality of ring interlocking sections 18 forming a system assembled with additional instruments fastened to each section. As described elsewhere herein, the ring interlocking sections 18 comprise a plurality of holes 16 that can be threaded, unthreaded, or a combination of threaded and unthreaded. The holes 16 can be used to attach any suitable instrument commonly used in the art, such as clamps 50 and fasteners 54. A plurality of clamps 50 can be anchored to one or more rods 52 to immobilize the system of ring interlocking sections 18. Fasteners 54 can be used to align and lock one or more pins 56. In some embodiments, a pair of fasteners 54 can be positioned across from each other on the ring interlocking sections 18 to secure one or more pins 56 between the pair of fasteners 54.

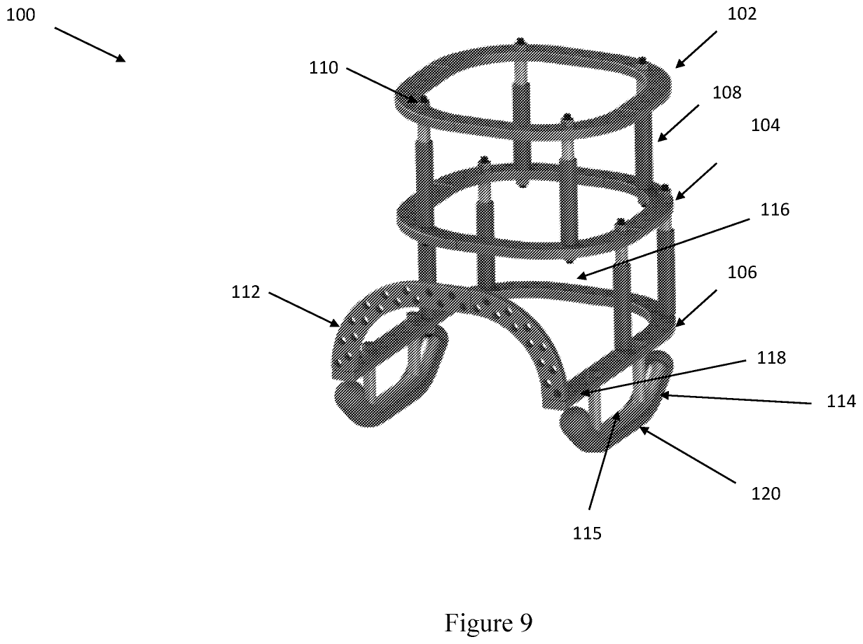

FIG. 9 shows an exemplary pre-built configuration of an external fixator system 100. The external fixator system 100 comprises a plurality of expandable fixator rings which are arranged at a distance to each other (Top expandable fixator ring 102, middle expandable fixator ring 104 and bottom expandable fixator ring 106) and at least one adjustable length strut 108, which is in connection with the expandable fixator rings. Fasteners 110, for example bolts or screws, can be used to secure the strut 108 relative to the expandable fixator rings. In one exemplary embodiment, the bottom expandable fixator ring 106 comprises also a rolling structure 114 to enable a user to walk around while wearing the external fixture system 100.

In one exemplary embodiment, as shown in FIG. 9, the external fixator system 100 comprises three expandable fixator rings. Top expandable fixator ring 102, middle expandable fixator ring 104 and bottom expandable fixator ring 106 serve as bearing elements for pins which are in connection with bony structure. The orientation as well as the distance between the top, middle and bottom expandable fixator ring 102, 104 and 106 thereby define the orientation and distance between fractured elements of the bony structure.

Adjustable length struts 108 each include a length-adjusting mechanism. In one exemplary embodiment, a telescopic strut can be used. A telescopic strut includes an axially extending rod having a series of circumferential grooves on an outer surface of the rod; an axially extending tube is provided for receiving the rod. The tube has a first end including a plurality of holes formed in a wall of the tube and a second end having a pin extending therethrough. A plurality of bearings are provided for extending through the holes in the first end of the tube. A sleeve having an eccentric bore therethrough is mounted around the tube first end for contacting the bearings. The eccentric bore has a major diameter allowing the bearings to be located outside the grooves of the rod and a minor diameter causing the bearings to be held within the grooves on the rod. A coupling element comprising a hollow tubular member is slidably mounted on an outer surface of the tube second end, the coupling element tubular member has two axially extending diametrically opposed slots for receiving the pin and an adjustment element is mounted on an outer surface of the coupling element tubular member for movement thereon in the axial direction. The adjustment member serves as a stop to limit the travel of the pin in the two slots. The adjustment member may be threaded and mounted on a mating thread on the coupling element tubular member.

A detent may be provided for holding the sleeve in a first position where the major diameter engages the bearings or a second position where the minor diameter engages the bearings. The means includes a spring biasing the sleeve towards the second position.

The grooves in the rod may be formed by a helical thread extending along the axial extent thereof so that rotation of the rod with the bearings engaged lengthens the strut. Alternately the grooves may be formed by a plurality of radial ridges. Preferably the bearings are at least partially retained within the holes of the leading end when contacted by the minor diameter of the sleeve.

In another exemplary embodiment, a telescopic strut for use with an external fixator may also comprise an axially extending rod, an axially extending tube moveably receiving the rod for varying the length of the rod and tube combination along a longitudinal axis. The tube has first and second ends, the second end having a pin extending therethrough. An adjustment system is mounted on the tube first end for adjusting the position of the rod in the tube. A connector element is provided having a tubular sleeve with a threaded outer surface mounted on an outer surface of the second end of the tube. The connector element can include a ball joint which can be locked when the connector is fixed in a hole in a ring of an external fixation frame. The sleeve has two axially extending diametrically opposed slots for receiving the pin and an adjustment element threadably mounted on a threaded portion of the tubular sleeve outer surface for axial movement along the axis. The adjustment element has an annular surface contacting the pin to limit the movement of the pin in the slots.

The rod is threaded and is mounted in the end of the tube such that relative rotation therebetween causes a length variation of the rod and tube combination along the longitudinal axis. The adjustment system may include radially moveable elements which selectively engage and disengage the threaded rod to allow axial sliding when disengaged and fine adjustment by the relative rotation of the tube and rod when engaged to vary the strut length.

The tubular sleeve is coupled to an opening in an external fixation ring by releasable connectors which allow rotation of the sleeve and tube about the axis of the rod and tube to vary the axial length of rod and tube when the radially moveable elements are engaged.

The two slots in the connector sleeve each have a first end and the adjustment element can move the pin into contact with the slot first end to prevent the pin from moving in the two slots. The sleeve outer surface includes markings showing the distance between the slot first end and the pin wherein the markings are in 1 mm increments.

The pin may include a pair of protruding ends for receiving a tool for rotating the tubular sleeve and tube relative to the rod for varying the length of the strut.

In another exemplary embodiment, a telescoping strut for an external fixator comprises a threaded rod, a tube threadably receiving the rod with the tube and rod extending along a longitudinal axis. The tube has a first end with a pin extending therethrough. An adjustment element is provided for fixing the relative axial position of the tube and rod; and a dynamization system mounted on the first end of the tube, the system comprising a sleeve slidably mounted on the tube with the pin extending through a pair of diametrically opposed slots on the sleeve such that the pin can move in the axial direction with respect to the slots; an adjustable stop element mounted on the sleeve movable to limit the level of the pin in the slots. The adjustable stop element is a nut mounted on a threaded outer surface of the sleeve.

In one exemplary embodiment, fasteners 110, for example bolts or screws, can be used to secure the strut 108 relative to the top, middle and bottom expandable fixator ring 102, 104, 106.

The bottom expandable fixator ring 106, in the particular embodiment shown, is generally “U” shaped with a posterior base 116 and anterior projections 118.

In one exemplary embodiment, the bottom expandable fixator ring 106 comprises also a rolling structure 114 to enable a user to walk around while wearing the external fixture system 100. Generally, the rolling structure 114 has an elongated main body 115 and an elongated ground-contacting rounded portion 120. The ground-contacting rounded portion 120 may include treads or other surface texture to increase friction with the ground. The ground-contacting rounded portion 120 may be integral with the main body 115, or may be otherwise coupled to the main body, for example by adhesive. As depicted in FIG. 9, two rolling structures 114 are used with the bottom expandable fixator rings 106 to provide stable contact with the ground. Also, the rolling structures 114 may be identical or may be similarly or symmetrically shaped. For example, the rolling structure 114 may include contours, which contours may be mirrored in a second rolling structure that is used with the bottom expandable fixator ring 106.

In one exemplary embodiment a half-ring 112 is coupled to the bottom expandable fixator ring 106. Although the term half-ring is used, it should be understood that the half-ring 112 is not limited to being half of a circular ring, but may take other shapes, such as generally arcuate shapes.

Among other benefits, the half-ring 112 closes the open bottom expandable fixator ring 106 such that the open bottom fixation ring 106 does not deflect or only minimally deflects, when fixation features (such as Kirschner wires or “K-wires”) are tensioned. Without half-ring 112, tensioning a member fixed at opposite ends of the open bottom expandable fixator ring 106, such as a K-wire, may tend to cause the open portions of bottom fixation ring 106 to deflect closer together. This deflection is limited by the rigid connection of the half-ring 112 to the bottom expandable fixator ring 106. The half-ring 112 may also include hinges at the ends where it is connected to the bottom expandable fixator ring 106. Hinges may be coupled to the half ring 112, for example by an adhesive, or may alternatively be integral with the half-ring 112. The hinged connector may be a compression fit or a snap fit, or may otherwise be fixed, for example by an adhesive.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An expandable external fixator device comprising:
a rigid frame having a ring-like shape and a plurality of pins positioned on the frame; and
a plurality of rigid segments, each segment having at least two slots connected to at least two pins of the frame and slidable between an inner position and an outer position;
wherein the device has a variable size defined by the position of the plurality of segments.

2. The device of claim 1, wherein each rigid segment comprises a plurality of through holes.

3. The device of claim 1, wherein the rigid frame has a ring-like shape that is substantially circular, ovoid, horseshoe, square, and rectangular.

4. The device of claim 1, wherein the position of each rigid segment is lockable by a mechanism selected from the group consisting of: a screw, a clamp, an adhesive, a weld, a ratchet, a knob, a latch, and a pin.

5. An expandable external fixator device comprising:
a plurality of interlocking rigid sections, each section having a slot positioned at a first end and a tab positioned at a second end;
wherein the tab of each section is sized to fit within the slot of each section, and wherein the device has a variable size defined by the position of each tab within each slot.

6. The device of claim 5, wherein each section comprises a plurality of through holes.

7. The device of claim 1, wherein the plurality of sections are configured to interlock to form a shape that is substantially circular, ovoid, horseshoe, square, and rectangular.

8. The device of claim 1, wherein the position of each rigid segment is lockable by a screw or clamp.

9. The device of claim 8, wherein the clamp comprises snap-in barbs configured to engage an interconnected tab and slot.

10. The device of claim 8, wherein the clamp comprises hinged pins configured to engage an interconnected tab and slot.

11. The device of claim 8, wherein the clamp comprises lockable pins configured to engage an interconnected tab and slot.

12. An external fixation system comprising:
one or more expandable fixator devices of claim 5; and
one or more length-adjustable struts;
wherein each expandable fixator device is securable to each other by the one or more length-adjustable struts.

13. The system of claim 12, further comprising one or more treads or rollers.
